(19) United States
(12) Patent Application Publication
(10) Pub. No.: US 2017/0342218 A1
(43) Pub. Date: Nov. 30, 2017

(54) Process for Preparing Tissue Regeneration Matrix

(71) Applicant: Integra LifeSciences Corporation, Plainsboro, NJ (US)

(72) Inventors: John E. Kemnitzer, West Windsor, NJ (US); Elaina Panas, Belle Mead, NJ (US); Ankur Gandhi, Metuchen, NJ (US); Sunil Saini, Monroe, NJ (US); Angana Kharge, Lawrence Township, NJ (US)

(21) Appl. No.: 15/606,035

(22) Filed: May 26, 2017

Related U.S. Application Data
(60) Provisional application No. 62/341,875, filed on May 26, 2016.

(51) Int. Cl. C08H 1/06; C07K 1/00; A61L 27/00; C07K 14/78; A61K 9/14

(52) U.S. Cl. CPC C08H 1/06; C07K 14/78; A61K 9/14; A61L 27/00; C07K 1/00

(57) ABSTRACT

The present invention is directed to a process for making a tissue regeneration matrix. The process comprises providing a collagen-tropoelastin dispersion; freeze-drying the dispersion to provide a porous freeze-dried matrix; and then crosslinking the porous freeze-dried matrix. The present invention is also directed to a tissue regeneration matrix prepared by the process.

### FIELD OF THE INVENTION

The invention generally relates to the field of tissue regeneration. More specifically, the invention relates to tissue regeneration matrices comprising collagen for use in, for example, wound care and dermal regeneration, and processes for making the matrices.

### BACKGROUND

Collagen is a natural body material useful in a wide range of medical applications. The incorporation of glycosaminoglycans (GAG) into collagen is recognized as providing for a matrix that allows for regeneration of primary tissues. Thus, collagen/GAG matrices represent a particularly useful family of collagen-containing materials.

U.S. Pat. No. 4,947,840, which is incorporated herein by reference in its entirety, discloses a biodegradable polymeric material for treating wounds, which acts as a scaffold and induces the wound to synthesize new tissue. The material preferably comprises Type-I collagen and glycosaminoglycan (GAG) in a covalently crosslinked sheet. The material has been shown to reduce contraction and scarring of dermal wounds when used in a sheet form and placed over wounds to promote regeneration.

U.S. Pat. No. 6,969,523, which is incorporated herein by reference in its entirety, describes compositions of cross-linked collagen and a glycosaminoglycan which retain characteristics rendering them useful as tissue engineering matrices or scaffolds following terminal sterilization. These biodegradable matrices are useful in a variety of biochemical applications, including, but not limited to, dermal replacement constructs. For example, the dermal replacement layer of the Integra® Dermal Regeneration Template (Integra LifeSciences Corporation, Plainsboro, N.J., U.S.A.) is comprised of a porous matrix of fibers of cross-linked bovine tendon collagen and the glycosaminoglycan chondroitin-6-sulfate. This commercially available bilayer membrane system for skin replacement is useful in the treatment of deep, partial-thickness, or full-thickness thermal injury to the skin such as third-degree burns. Following application to the wound, the bilayer functions as an artificial skin that provides immediate post-excisional wound homeostasis, facilitating patient recovery and relieving metabolic stress.

Although some tissue regeneration materials are available commercially, there remains a need for tissue regeneration materials that have improved physical properties and effectiveness for tissue regeneration.

### SUMMARY

In accordance with an aspect of the present invention, a process for making tissue regeneration matrices is provided. The process comprises: a) providing a collagen- tropoelastin dispersion; (b) freeze-drying the collagen-tropoelastin dispersion to provide a porous freeze-dried matrix; and (c) crosslinking the porous freeze-dried matrix. The dispersion may comprise about 70 to about 95% collagen and about 5% to 30% tropoelastin. The dispersion may comprise about 70 to about 99% collagen and about 1% to 30% tropoelastin. The dispersion may comprise about 85% to about 95% collagen and about 5% to 15% tropoelastin.

In accordance with another aspect of the present invention, a tissue regeneration matrix is provided. The tissue regeneration matrix is prepared by the process of the present invention described above.

In accordance with a further aspect of the present invention, a tissue regeneration matrix comprising collagen and elastin is provided, wherein the elastin is generated by crosslinking tropoelastin in the presence of collagen, in vitro. The matrix may further comprise tropoelastin.

In accordance with yet another aspect of the present invention, a tissue regeneration matrix comprising collagen and tropoelastin is provided. The tissue regeneration matrix is prepared by providing a mixture of collagen and tropoelastin, and crosslinking the mixture. The matrix may further comprise elastin.

These and other features and advantages of the invention or certain embodiments of the invention will be apparent to those skilled in the art from the following disclosure and description of exemplary embodiments.

### DETAILED DESCRIPTION OF EXEMPLARY EMBODIMENTS

The process of the present invention for preparing a tissue regeneration matrix comprises: a) providing collagen-tropoelastin dispersion; (b) freeze-drying the collagen-tropoelastin dispersion to provide a freeze-dried matrix; and (c) crosslinking the freeze-dried matrix. The freeze-dried matrix may be porous, and it may remain porous after being cross-linked. The process may additionally comprise a step of dehydrothermal treatment of the porous freeze-dried matrix, and may further comprise a step of applying a synthetic polymeric layer, such as a silicone layer.

Embodiments of the present invention are based on the discovery that tropoelastin, for example, recombinant human tropoelastin (rhTE), may be solubilized and readily incorporated into a collagen dispersion, presumably resulting in an ionic binding of the collagen dispersion. This dispersion is able to be lyophilized and dehydrothermally (DHT) processed resulting in a matrix with the appropriate porosity properties of a dermal regeneration matrix. This matrix may be further stabilized (i.e., crosslinked using wet or dry methods), without being bound by any theory, presumably locking in covalently the rhTE into the matrix structure. It is hypothesized that polymerization of the rhTE has also occurred during both the DHT and solution crosslinking steps, i.e. chemical crosslinking steps. The resulting product may be sterilizable by E-beam irradiation or ethylene oxide irradiation.

Collagen is a major protein component of bone, cartilage, skin, and connective tissue in animals. Collagen occurs in several types, having differing physical properties. The most abundant types are Types I, II and III. In an exemplary embodiment of the present invention, Type I collagen is used in the process of the present invention.

Collagen derived from any source is suitable for use in the compositions of the present invention, including insoluble collagen, collagen soluble in acid, in neutral or basic aqueous solutions, as well as those collagens that are commercially available. Typical animal sources for collagen include but are not limited to recombinant collagen, fibrillar collagen from bovine, porcine, ovine, cuprine and avian sources as well as soluble collagen from sources such as cattle bones and rat tail tendon.

Other types of molecules that can be used in combination with collagen during the manufacturing process include, but are not limited to, chitin, chitosan, fibronectin, laminin, decorin, and the like, or combinations thereof.

Elastin is an extracellular matrix protein that is found in connective tissues and other tissues such as skin. Elastin has elastic properties. Tropoelastin is the monomeric form of elastin. The monomer polypeptides form elastin when cross-linked. In vivo, tropoelastin monomers are cross-linked by lysyl oxidase to form elastin.

U.S. Pat. No. 6,808,707, which is incorporated herein by reference in its entirety, describes that the genes encoding tropoelastin have been cloned from a variety of organisms including human and non-human organisms, and that a variety of different isoforms of human tropoelastin are produced in nature (by alternative splicing). The gene and expression of human tropoelastin have been described in Indik et al (1990) Archives of Biochemistry and Biophysics, 280(1), 80-86; Martin et al. (1995) Gene, 154, 159-166; and U.S. Pat. Nos. 6,232,458 and 7,700,126, which are all incorporated herein by reference in their entireties. In addition, the tropoelastin protein may be modified, as compared with naturally occurring tropoelastin protein, either chemically or genetically in vivo or in vitro. Tropoelastin can be prepared recombinantly or by chemical synthesis. Tropoelastin in any form and from any source, including full length tropoelastin, isoforms of tropoelastin, genetically engineered tropoelastin constructs, fragments and derivatives of tropoelastin, or a combination thereof, may be used in the process of the present invention for preparing tissue generation matrices.

The collagen-tropoelastin dispersion comprises about 70 to about 99% collagen and about 1% to about 30% tropoelastin. In certain embodiments, the dispersion comprises about 50 to about 99% collagen and about 1% to about 50% tropoelastin. The collagen-tropoelastin dispersion comprises about 80% to about 90% collagen and about 10% to about 20% tropoelastin. The collagen-tropoelastin dispersion comprises about 85% to about 95% collagen and about 5% to about 15% tropoelastin. It is to be understood that other ingredients may be included in the collagen-tropoelastin dispersion outside of the ranges described.

For preparation of a collagen-tropoelastin dispersion, collagen or tropoelastin may be added to the dispersion in any order. In exemplary embodiments, a collagen dispersion is prepared first, and then tropoelastin is manually mixed into the collagen dispersion to yield a collagen-tropoelastin dispersion, in a 97% collagen/3% tropoelastin (w/w) ratio, or a 95% collagen/5% tropoelastin (w/w) ratio or a 90% collagen/10% tropoelastin (w/w) ratio or a 85% collagen/15% tropoelastin (w/w) ratio.

The freezing process may be followed by sublimation of ice crystals to produce the scaffold pore structure. Dehydrothermal treatment can be performed at an elevated temperature, for example under vacuum pressure for a sufficient period of time.

As used herein for purposes of the present invention, the terms “matrix”, “matrices”, “scaffold” or “scaffolds” refer to a construct of natural or synthetic biomaterials, particularly collagens and their derivatives that can be used in a composite with other materials, which are used in vivo and in vitro as structural supports for cells and tissues, frameworks for tissue formation and regeneration, surfaces for cell contact, or delivery systems for therapeutics. Thus, by the term matrix or scaffold, it is meant to include load-bearing materials, bulking agent and fillers, and physiological barriers as well as frameworks for tissue formation and delivery systems for cells, biomolecules, drugs and derivatives thereof.

Covalent cross-linking can be achieved by various coupling or cross-linking reagents, some of which are suitable for biological applications. One suitable chemical method for covalently cross-linking collagen/tropoelastin matrices is known as aldehyde cross-linking. In this process, the materials are contacted with aqueous solutions of aldehyde, which serve to cross-link the materials. Suitable aldehydes include formaldehyde, glutaraldehyde and glyoxal. The preferred aldehyde is glutaraldehyde because it yields a desired level of cross-link density more rapidly than other aldehydes and is also capable of increasing the cross-link density to a relatively high level. When glutaraldehyde is used as the cross-linking agent, it is preferred that nontoxic concentrations of greater than about 0.25% be used. Other chemical techniques that are suitable for increasing cross-link density in the present invention include carbodiimide coupling, azide coupling, and diisocyanate cross-linking, as well as crosslinking with polyethylene glycol (PEG).

It is contemplated that the collagen-tropoelastin matrix scaffold may be in a single layer configuration, without a silicone layer, or in a multiple layer configurations, including one or more layers of biocompatible materials. In an exemplary embodiment, a collagen- tropoelastin matrix scaffold in a bilayer configuration, with a silicone layer, is provided for promoting dermal regeneration. The silicone layer may be applied to the collagen-tropoelastin matrix prior to cross-linking. This layer is applied in accordance with well-known techniques. An exemplary method for application of the silicone layer to the matrix or scaffold is set forth in Example 6. The silicone layer can be added prior to sterilization or after sterilization at the point of care.

The matrices of the invention can further comprise bioactive molecules effective to achieve a desired result. Suitable bioactive molecules include, but are not limited to, growth factors, anti-inflammatory agents, wound healing agents, anti-scarring agents, antimicrobial agents (for example, silver), cell-adhesion peptides including Arg-Gly-Asp (RGD) containing peptides, nucleic acids, nucleic acid analogues, proteins, peptides, amino acids, and the like, or combinations thereof.

Pharmacologically active agents such as VEGF (vascular endothelial cell growth factor), FGF (the fibroblast growth factor family of proteins), TGF.beta. (transforming growth factor B), hepatocyte growth factor (HGF), platelet factor 4 (PF4), PDGF (platelet derived growth factor), EGF (epidermal growth factor), NGF (nerve growth factor), BMP (bone morphogenetic protein family), coagulation factors such as one of the vitamin K-dependent coagulant factors, such as Factor II/IIa, Factor VIINIIa, Factor IX/IXa or Factor X/Xa. Factor V/Va, VIIINIIIa, Factor XI/XIa, Factor XII/XIIa, Factor XIII/XIIIa, and mixtures thereof may also be used. Antimicrobials, antibiotics, antifungal agents, hormones, enzymes, enzyme inhibitors, and mixtures thereof can also be incorporated in the compositions of the instant invention and subsequently delivered to the wound site.

Sterilization can be performed by any method conventional in the art, although electron beam irradiation is a preferred method, especially when a silicone layer is present.

The incorporation of tropoelastin into the collagen matrix provides improved properties and effectiveness compared to the original collagen matrix, including increased elasticity and mechanical properties, earlier revascularization and dermal regeneration, faster re-epithelization, and reduced scar and contracture.

The collagen-tropoelastin matrix scaffold is porous and biodegradable. The average pore size is within the range of about 100 μm to about 600 μm. The pore volume is preferably within the range of 75-95%.

The matrices as described herein may be applied to the fields of wound care (chronic and acute), burn care (2nd and 3rd degree), dural repair/regeneration, muscle repair/regeneration, nerve repair/regeneration, tendon repair/regeneration, abdominal wall repair/reconstruction, breast reconstruction and bone repair/regeneration.

These matrices and scaffolds are useful in medical and surgical applications, for the regeneration of dermal and sub-dermal tissue. Specifically they can be used for dermal regeneration after excision of burns, scars, and other injuries or for filling of tissue, for example, after excisions of tumors or for cosmetic application such as augmentation of tissue. In this embodiment, the matrix or scaffold is applied to or implanted within a subject at or near the site of the excision or the site where augmentation of tissue is required. Methods for application of such matrices or scaffold are well known by those of skill in the art.

Other applications of collagen-tropoelastin matrix scaffolds of the present invention may include, but are not limited to, surgical sutures, blood vessel grafts, catheters and, in general, the fabrication of surgical prostheses. Additionally, these matrices or scaffolds are useful in the fabrication of artificial organs that pump blood, such as artificial kidneys, and blood compatible equipment such as blood oxygenators, as well as in the fabrication of miscellaneous equipment for the handling and storage of blood such as pumps, tubes and storage bags.

EXAMPLES The following examples describe the manufacture of a collagen-tropoelastin matrix in a bilayer configuration with a silicone layer to promote dermal regeneration. The tropoelastin is added to the collagen dispersion in a 97% collagen/3% tropoelastin (w/w) ratio, a 95% collagen / 5% tropoelastin (w/w) ratio, a 90% collagen/10% tropoelastin (w/w) ratio or a 85% collagen / 15% tropoelastin (w/w) ratio. The examples are specific embodiments of the present invention but are not intended to limit it.

### Example 1

### Preparation of Collagen Dispersion

A collagen dispersion is produced using Type I collagen (0.5% w/w) purified from bovine tendon (Integra Life Sciences, Plainsboro, N.J.) in 0.05M acetic acid (Glacial Acetic Acid, USP, CAS No. 64-19-7) solution.

A 0.05M acetic acid solution was prepared by combining 50-55 kg of Water for Injection (WFI) with 330 g acetic acid, and continuously adding WFI while mixing to reach 110 kg (pH 3.2, 0.5-2 ° C.).

A collagen dispersion in 0.05M acetic acid is prepared by adding 500 g (dry weight) purified Type I collagen (Integra LifeSciences, Plainsboro, N.J.) into the 0.05M acetic acid solution and mixing in a stainless steel vessel (9500 RPM) at 10° C. for 30 minutes.

The dispersion is then degassed for 30 minutes to remove air bubbles.

### Example 2

### Preparation of Collagen-Tropoelastin Dispersion

GMP-grade recombinant human tropoelastin isoform SHELA26A (Synthetic Human Elastin without domain 26A) corresponding to amino acid residues 27-724 of GenBank entry AAC98394 (gi 182020) is provided by Elastagen Pty Ltd., and manually mixed into the collagen dispersion using a 90% collagen/10% tropoelastin (w/w) ratio to yield a dispersion.

A mass of 0.6434 g tropoelastin is manually mixed into 1156 g collagen dispersion and subsequently mixed by stir bar for 20 minutes at 20° C., then degassed for 30 minutes under vacuum to remove air bubbles.

A mass of 210 g of collagen-tropoelastin dispersion is pipetted into a stainless steel tray (inner tray dimensions 28.5 cm length×23.3 cm width×2.8 cm depth; Grade 316 SS) for lyophilization. The dispersion is freeze-dried (Millrock Magnum Max85 Freeze Dryer, Model MX85B10, Kingston, N.Y.) by first cooling from 10° C. to −35° C. at a rate of 0.25° C./min and then held at constant temperature for 2 hours.

### Example 3

### Alternative Preparation of Collagen-Tropoelastin Dispersion

GMP-grade recombinant human tropoelastin isoform SHELA26A (Synthetic Human Elastin without domain 26A) corresponding to amino acid residues 27-724 of GenBank entry AAC98394 (gi 182020) is provided by Elastagen Pty Ltd., and manually mixed into the collagen dispersion using a 90% collagen/10% tropoelastin (w/w) ratio to yield a dispersion.

A mass of 0.6 g tropoelastin is added into a centrifuge tube. A small amount (<30 ml) of sterile PBS is pipetted into the centrifuge tube to wet the tropoelastin. The tube is quickly transferred to 4° C. and allowed to stand overnight. After 24 hours, the solution is pipetted out and manually mixed into 1200 g collagen dispersion and subsequently mixed by stir bar for 20 minutes, then degassed for 30-60 minutes under vacuum to remove air bubbles. Temperature is maintained between 2 and 9° C. during both the mixing and degassing steps.

For solutions that incorporate high amounts of tropoelastin (75% collagen/25% tropoelastin) tropoelastin in PBS is left to stand at 4° C. for 36-48 hours to come into solution. Following dissolution, the tropoelastin-PBS solution is added to 1200 g collagen-dispersion and mixed by stir bar for 20 minutes. The homogenized solution is degassed for 30-60 minutes under vacuum to remove air bubbles. Temperature of the solution is maintained between 2 and 9° C. during both the mixing and degassing steps.

A mass of 210 g of collagen-tropoelastin dispersion is pipetted into a stainless steel tray (inner tray dimensions 28.5 cm length×23.3 cm width×2.8 cm depth; Grade 316 SS) for lyophilization. The dispersion is freeze-dried (Millrock Magnum Max85 Freeze Dryer, Model MX85B10, Kingston, N.Y.) by first cooling from 10° C. to −20° C. at a rate of 0.1° C./min and then held at constant temperature for 2 hours.

### Example 4

### Lyophilization and Dehydrothermal Treatment

The freezing process is followed by sublimation of ice crystals to produce the scaffold pore structure. The sublimation process is performed at 200 mtorr vacuum pressure and under a temperature ramp rate of 0.83° C./min for the first 3 hours from −35° C. to −20° C., 0.013° C./min for 6.5 hours up to −15° C. and held at constant temperature for 1 hour, 0.03° C./min for the next 5 hours up to −5° C., and 0.125° C./min for 4 hours up to 25° C.

Dehydrothermal treatment is then performed at 105° C. under 200 mtorr vacuum pressure for 24 hours to generate dry collagen-tropoelastin matrix scaffolds.

### Example 5

### Alternative Lyophilization and Dehydrothermal Treatment

The freezing process is followed by sublimation of ice crystals to produce the scaffold pore structure. The sublimation process is performed at 710 mtorr vacuum pressure and under a temperature ramp rate of 0.008° C./min for the first 9.9 hours from −20° C. to −15° C., held at constant temperature for 30 mins, 0.033° C./min for 5 hours up to −5° C., 0.125° C./min for the next 4 hours up to −25° C. at 50 mtorr vacuum pressure, and held constant at 25° C. for 1 hour.

Dehydrothermal treatment was then performed at 105° C. under 50 mtorr vacuum pressure for 20 hours to generate dry collagen-tropoelastin matrix scaffolds.

### Example 6

### Crosslinking the Matrix

Chemical crosslinking is performed in a 0.5% (w/w) glutaraldehyde in 0.05M acetic acid solution.

Dry collagen-tropoelastin matrix scaffolds are soaked in 0.05M acetic acid (Glacial Acetic Acid, Cat No. 338826, Sigma-Aldrich, St. Louis, Mo.) followed by subsequent exposure to 0.5% glutaraldehyde (50% Glutaraldehyde solution, Cat No. 340855, Sigma-Aldrich, St. Louis, Mo.) in a 0.05M acetic acid solution and then performing a series of rinse steps to remove residual glutaraldehyde.

Dry scaffolds are lined single-sided with a polyethylene sheet for support, placed into meshed polypropylene frames and stacked horizontally in a Nalgene bin. A 0.05M acetic acid solution is prepared by adding 60 ml acetic acid to 19.94L deionized water (pH 3.2; room temperature) and mixed for 30 minutes.

Using a peristaltic pump and tubing, the 0.05M acetic acid solution is pumped into the basin until scaffolds are completely submerged and soaked 17 hours. In a separate bin, the 0.5% glutaraldehyde crosslinking solution is prepared composed of 0.5% (w/w) glutaraldehyde in 0.05M acetic acid solution. Specifically, 198m1 glutaraldehyde solution (50%) is added to an acetic acid solution containing 60m1 acetic acid and 19.742L deionized water and is mixed by stir bar for 20 minutes.

Following the overnight soak, the acetic acid solution is removed from the original Nalgene bin using a peristaltic pump and is replaced with the 0.5% glutaraldehyde crosslinking solution. Scaffolds are soaked in the glutaraldehyde crosslinking solution for 22.5 hours.

Crosslinking solution is removed and is replaced with deionized water for 30 minutes to rinse residual glutaraldehyde from scaffolds. Four additional rinse steps with deionized water are performed for 30 minutes each, with the final rinse lasting for 15 hours.

Following the final rinse, deionized water is removed and replaced with 10 mM sodium phosphate buffer solution (200 g 1M NaH₂PO₄.H₂O diluted in 19.8L deionized water, followed by subsequent addition of 50 mL 1M NaOH, and incremental addition of 1M NaOH to achieve pH of 6.5) and soaked for 30 minutes prior to packaging.

### Example 7

### Alternative Method of Crosslinking the Matrix

Chemical crosslinking is performed in a 0.5% (w/w) glutaraldehyde in 0.05M acetic acid solution.

Dry collagen-tropoelastin matrix scaffolds are soaked in 0.05M acetic acid (Glacial Acetic Acid, Cat No. 338826, Sigma-Aldrich, St. Louis, Mo.) followed by subsequent exposure to 0.5% glutaraldehyde (50% Glutaraldehyde solution, Cat No. 340855, Sigma-Aldrich, St. Louis, Mo.) in a 0.05M acetic acid solution and then performing a series of rinse steps to remove residual glutaraldehyde.

Dry scaffolds are lined single-sided with a polyethylene sheet for support, placed into meshed polypropylene frames and stacked horizontally in a Nalgene bin. A 0.05M acetic acid solution is prepared by adding 60 ml acetic acid to 19.94L deionized water (pH 3.2; room temperature) and mixed for 30 minutes.

Using a peristaltic pump and tubing, the 0.05M acetic acid solution is pumped into the basin until scaffolds are completely submerged and soaked for 1 hour. In a separate bin, the 0.5% glutaraldehyde crosslinking solution is prepared composed of 0.5% (w/w) glutaraldehyde in 0.05M acetic acid solution. Specifically, 198 ml glutaraldehyde solution (50%) is added to an acetic acid solution containing 60 ml acetic acid and 19.742L deionized water and is mixed by stir bar for 20 minutes.

Following the hour long soak, the acetic acid solution is removed from the original Nalgene bin using a peristaltic pump and is replaced with the 0.5% glutaraldehyde crosslinking solution. Scaffolds are soaked in the glutaraldehyde crosslinking solution for 22±1 hours.

Crosslinking solution is removed and is replaced with deionized water for 90 minutes to rinse residual glutaraldehyde from scaffolds. Four additional rinse steps with deionized water are performed for 90 minutes each, with the final rinse lasting for 15±3 hours.

Following the final rinse, deionized water is removed and replaced with 10 mM sodium phosphate buffer solution (200 g 1M NaH₂PO₄.H₂O diluted in 19.8L deionized water, followed by subsequent addition of 50 mL 1M NaOH, and incremental addition of 1M NaOH to achieve pH of 6.5) and soaked for 30 minutes prior to packaging. Scaffolds are cut to size, soaked in PBS solution for 2 minutes and then packaged.

### Example 8

### Alternative Method of Crosslinking the Matrix

Vapor crosslinking is performed with formaldehyde in a crosslinking chamber. Dry collagen-tropoelastin matrix scaffolds are placed in a hanging frame and secured with clips. 25% formaldehyde solution (25%) is prepared from a stock solution (37% Formaldehdye, Sigma-Aldrich, St. Louis, Mo.) by adding 675 ml stock formaldehyde to 325 ml DI water at room temperature. The matrices are vapor crosslinked for 90 minutes and allowed to vent for 18-24 hours. The crosslinked matrices are cut to size and packaged in pouches appropriate for sterilization.

### Example 9

### Sterilization

Sterilization is performed by electron beam irradiation (17.5-35 kGy). Scaffolds are removed from mesh frames with polyethylene sheet, and are sandwiched in between two polyethylene sheets to aid in product handling. Scaffolds are packaged in a foil moisture-barrier pouch and are terminally sterilized by electron beam irradiation (17.5-25 kGy).

### Example 10

### Alternative Method of Sterilization

Sterilization is performed by electron beam irradiation (17.5-35 kGy) or with ethylene oxide. Wet-crosslinked scaffolds are removed from mesh frames with polyethylene sheet, and are sandwiched in between two polyethylene sheets to aid in product handling. Scaffolds are packaged in a foil moisture-barrier pouch and are terminally sterilized by electron beam irradiation (17.5-25 kGy). Dry crosslinked scaffolds are packaged in either breathable Tyvek pouches or foil pouches. They are terminally sterilized using either EO or Ebeam methods.

### Example 11

### Application of Silicone Layer

A silicone layer could be added prior to sterilization or after sterilization at the point of care. A 0.26″ silicone layer is added via feeding dry sponges or matrices through a silicone dispensing pump with silicone coated polyethylene sheets. Alternatively, a silicone layer may be added manually at the time of application.

Given the benefit of the above disclosure and description of exemplary embodiments, it will be apparent to those skilled in the art that numerous alternative and different embodiments are possible in keeping with the general principles of the invention disclosed here. Those skilled in this art will recognize that all such various modifications and alternative embodiments are within the true scope and spirit of the invention. The appended claims are intended to cover all such modifications and alternative embodiments. It should be understood that the use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc.

What is claimed is:

1. A process for making a tissue regeneration matrix, comprising:
a) providing a collagen-tropoelastin dispersion;
(b) freeze-drying the collagen-tropoelastin dispersion to provide a porous freeze-dried matrix; and
(c) crosslinking the porous freeze-dried matrix.

2. The process of claim 1, wherein the dispersion comprises about 50 to about 99% collagen and about 1% to about 50% tropoelastin.

3. The process of claim 1, wherein the dispersion comprises about 70 to about 95% collagen and about 5% to about 30% tropoelastin.

4. The process of claim 1, wherein the dispersion comprises about 80 to about 95% collagen and about 5% to about 20% tropoelastin.

5. The process of claim 1, wherein the crosslinking step is performed by subjecting the matrix to an aldehyde solution.

6. A tissue regeneration matrix prepared by the process of claim 1.

7. A tissue regeneration matrix comprising collagen and elastin, wherein the elastin is generated by crosslinking tropoelastin in the presence of collagen in vitro.

8. A tissue regeneration matrix comprising collagen and tropoelastin, prepared by a) providing a mixture of collagen and tropoelastin, and b) crosslinking the mixture.

9. The process of claim 1 wherein the crosslinking step is performed by subjecting the matrix to a formaldehyde vapor.

10. The process of claim 1 further comprising the step of sterilizing the tissue regeneration matrix using electron beam irradiation or ethylene oxide.

11. The process of claim 1 further comprising the step of sterilizing the tissue regeneration matrix as a dry matrix using electron beam irradiation or ethylene oxide.

12. The process of claim 1 further comprising the step of sterilizing the tissue regeneration matrix as a wet matrix using electron beam irradiation.
